(19) United States
(12) Patent Application Publication
(10) Pub. No.: US 2021/0116448 A1
(43) Pub. Date: Apr. 22, 2021

(54) METHOD FOR MEASURING FIBRINOGEN CONCENTRATION IN BLOOD SAMPLE AND NANOPARTICLES FOR SAME

(71) Applicant: KOREA UNIVERSITY RESEARCH AND BUSINESS FOUNDATION, Seoul (KR)

(72) Inventors: Dae Sung YOON, Seoul (KR); In Su ` KIM, Seoul (KR); Do Hyung KWON, Seoul (KR); Dong Tak LEE, Seoul (KR); Sang Won LEE, Seoul (KR); Gyu Bok LEE, Seoul (KR); Gyu Do LEE, Namyangju-si (KR)

(73) Assignee: KOREA UNIVERSITY RESEARCH AND BUSINESS FOUNDATION, Seoul (KR)

(21) Appl. No.: 17/047,549

(22) PCT Filed: Apr. 8, 2019
(86) PCT No.: PCT/KR2019/004132

§ 371 Date: Oct. 14, 2020

(30) Foreign Application Priority Data
Apr. 17, 2018 (KR) 10-2018-0044461

(51) Int. Cl. G01N 33/543; G01N 33/68; G01N 21/31

(52) U.S. Cl. CPC G01N 33/54346; G01N 2021/3133; G01N 21/31; G01N 33/68

(57) ABSTRACT

The present disclosure relates to a method for measuring fibrinogen concentration in a blood sample, which enables measuring of the concentration of the fibrinogen protein present in a blood sample from the human body. The method for measuring fibrinogen concentration of the present disclosure is convenient because an enzyme is not used. In addition, an error due to a factor affecting factor affecting in-vivo enzyme activity does not occur and measuring time is decreased since measurement for reference plasma is unnecessary. Therefore, the method achieves superior accuracy, precision and reproducibility as compared to the existing technologies and can be usefully employed for measuring fibrinogen concentration in a blood sample.

### TECHNICAL FIELD

The present disclosure relates to a method for measuring fibrinogen concentration in a blood sample, more particularly to a method for measuring fibrinogen concentration in a blood sample, which enables measuring of the concentration of the fibrinogen protein present in a blood sample from the human body, and nanoparticles for the method.

### BACKGROUND ART

Human has many sensory organs and senses various stimuli from outside, including the five senses, pain, temperature, etc. These functions are performed by sensory organs in organisms, and by sensors in machines or appliances. Thus, a biosensor can be thought of as a system which uses a biological element or mimics a biological system when acquiring information from an object to be detected and converts the information to recognizable signals such as color, fluorescence or electrical signals. A variety of types of biosensors can be configured with an analyte and a biological element, a signal transducer, etc. immobilized on the sensor. As methods for signal transduction by the signal transducer, various physical and chemical techniques including electrochemical, thermal, optical and mechanical methods are used.

A glucose sensor developed in 1962 by Clark using a dialysis membrane for measuring glucose is known as the first biosensor. In the early stage, most biosensors were prepared by immobilizing enzymes on signal-transducing elements. But, recently, with the rapid development of molecular biology, sensors prepared using monoclonal antibodies, antibody-enzyme conjugates, etc. are being developed and used. In addition, for high-throughput processing of a large quantity of genetic information, researches are being conducted actively on chip sensors such as DNA chips or protein chips, and many efforts are being focused on the development of high-tech sensors wherein molecular biology technology, nanotechnology and information and communications technology are integrated.

The biosensor is used to quantitatively or qualitatively analyze physical or chemical reactions depending on the presence or concentration of an analyte using electrical, optical or other methods. Use for clinical diagnosis or medical treatment accounts for about 90% of the whole biosensor market, and other applications include industrial uses such as detection of environmentally related materials such as environmental hormones, BOD in wastewater, heavy metals and agrichemicals, detection of harmful materials included in food such as agrichemical residues, antibiotics, pathogens or heavy metals for food safety testing, military use for detecting biochemical weapons such as sarin or Bacillus anthracis, control of growth condition of microorganisms in fermentation processes, monitoring of specific chemicals generated in chemical/petrochemical, pharmaceutical or food processing processes and academic uses such as the kinetic analysis of binding with biomaterials.

However, the currently available biosensor technologies require a large quantity of sample for recognition of the biomaterial to be detected. In addition, they are complicated in that very complex steps of analyte addition, signal generation, signal amplification, analysis result interpretation, etc. are necessary for sample analysis and very high cost is required for actual application.

Fibrinogen, which is also known as clotting factor I, plays a critical role in hemostasis and wound healing. Fibrinogen is a glycoprotein with an apparent molecular weight of 340 kDa, which is synthesized in the liver. It is composed of two dimers, each consisting of three pairs of different polypeptide chains called Aα, Bβ and γ, joined together by disulfide bridges. It circulates in bloodstream with a concentration of about 150-400 μg/mL. When the blood vessel is damaged, blood platelets are activated and plugs are formed. Fibrinogen is involved in primary hemostasis by contributing to crosslinking with the activated blood platelets.

At the same time, the activation of the coagulation cascade is initiated. At the end point, fibrinogen is converted to fibrin by proteolytic release of fibrinopeptide A and fibrinopeptide B, at a slower rate, by thrombin. The soluble fibrin monomers are assembled into double-stranded twisted fibrils. Subsequently, the fibrils are arranged in a lateral manner, resulting in thicker fibers. These fibers are then crosslinked by FXIIIa to a fibrin network, which stabilizes the blood platelet plugs via interaction of the fibrins with activated blood platelets, resulting in a stable clot.

Currently, fibrinogen is measured by measuring the change in optical characteristics depending on aggregation of fibrinogen using a fibrinogen-aggregating enzyme (Clauss assay and prothrombin time-derived assay). However, these technologies require the measurement of a solution with a known fibrinogen concentration (plotting of a calibration curve) for measurement of the fibrinogen concentration of a sample.

This method has the problem that the storage and quantitative addition of the enzyme are relatively difficult. This results in measurement errors. In addition, for measuring the fibrinogen concentration of a sample, the sample should be measured after making measurements (plotting a calibration curve) for a solution with a known fibrinogen concentration (reference plasma) while diluting the solution. Accordingly, the measurement is complicated and different results are obtained for different reference plasma available from different companies.

The inventors of the present disclosure have made consistent efforts to measure fibrinogen concentration in a blood sample without using an enzyme and reference plasma. As a result, they have completed the present disclosure by identifying that use of gold nanoparticles having optical properties, which are coated with a cell membrane capable of binding fibrinogen on the surface thereof, causes the gold nanoparticles to aggregate in proportion to concentration due to the structural property of fibrinogen dimers and the aggregation changes the optical properties of the gold nanoparticle, allowing the measurement of the concentration of fibrinogen, and that the cell membrane blocks the access of molecules other than fibrinogen, thereby remarkably reducing reactivity to other proteins in blood.

### DISCLOSURE

### Technical Problem

The present disclosure is directed to providing a method for measuring fibrinogen concentration in a blood sample, which enables measuring of the concentration of the fibrinogen protein present in a blood sample from the human body.

The present disclosure is also directed to providing nanoparticles for the method for measuring fibrinogen concentration in a blood sample.

### Technical Solution

The present disclosure provides nanoparticles for measuring fibrinogen concentration, having a material binding specifically to fibrinogen coated on the surface thereof.

The present disclosure also provides a method for measuring fibrinogen concentration in a blood sample, which includes:

(1) a step of contacting nanoparticles having a material binding specifically to fibrinogen coated on the surface thereof with a blood sample; (2) a step of inducing aggregation of the nanoparticles through binding of the material coated on the surface of the nanoparticles and fibrinogen in a blood sample; (3) a step of measuring the spectroscopic property of the nanoparticles; and (4) a step of calculating fibrinogen concentration in the blood sample using the measured spectroscopic property of the nanoparticles.

### Advantageous Effects

A method for measuring fibrinogen concentration of the present disclosure is convenient because an enzyme is not used. In addition, an error due to a factor affecting factor affecting in-vivo enzyme activity does not occur and measuring time is decreased since measurement for reference plasma is unnecessary. Therefore, the method achieves superior accuracy, precision and reproducibility as compared to the existing technologies and can be usefully employed for measuring fibrinogen concentration in a blood sample.

### BRIEF DESCRIPTION OF DRAWINGS

FIG. 1 schematically illustrates a method for measuring fibrinogen concentration in a blood sample of the present disclosure.

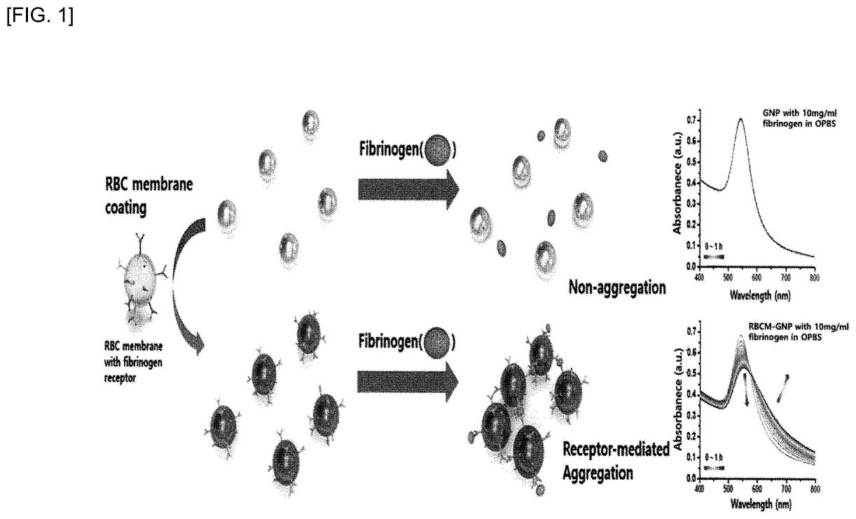

FIG. 2 shows the TEM images of gold nanoparticles and red blood cell membrane-coated gold nanoparticle of the present disclosure.

FIG. 3 shows the image of a tube in which red blood cells were purified from whole blood in the present disclosure.

FIG. 4 shows the change in the optical properties (left) and particle size (right) of gold nanoparticles before and after coating of a red blood cell membrane in the present disclosure.

FIG. 5 shows a result of measuring the fibrinogen spectra of red blood cell membrane-coated gold nanoparticles (top) and the fibrinogen spectra of gold nanoparticles (bottom) in the present disclosure.

FIG. 6 shows a result of measuring the fibrinogen spectra of red blood cell membrane-coated gold nanoparticles (top) and the fibrinogen spectra of gold nanoparticles (bottom) in the present disclosure (650 nm/542 nm, 609 nm/542 nm and 700 nm/542 nm).

FIG. 7 shows a result of measuring the spectra of human serum albumin (left) and γ-globulin (right) of red blood cell membrane-coated gold nanoparticles in the present disclosure.

FIG. 8 shows a 96-well plate using a multi-plate reader (left) and data measured using the 96-well plate (right) in the present disclosure.

FIG. 9 shows a result of measuring the fibrinogen spectra of mononuclear leukocyte membrane-coated gold nanoparticles in the present.

FIG. 10 shows the wavelength ranges in which light can be absorbed by different nanoparticles.

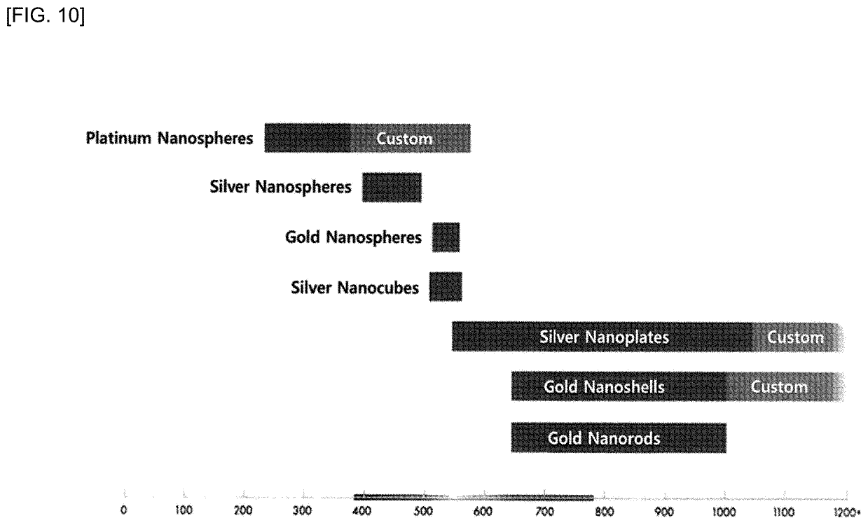

### BEST MODE

In the present disclosure, a cell membrane capable of binding fibrinogen was coated on the surface of gold nanoparticles having optical properties. It was confirmed that the cell membrane-coated gold nanoparticles aggregate with each other in the presence of fibrinogen in proportion to the concentration of fibrinogen. It was also confirmed that the aggregation of the gold nanoparticles changes the optical properties of the gold nanoparticles and, thereby, enables the measurement of the concentration of fibrinogen.

Accordingly, in an aspect, the present disclosure may provide a method for measuring fibrinogen concentration in a blood sample, which includes: (1) a step of contacting nanoparticles having a material binding specifically to fibrinogen coated on the surface thereof with a blood sample; (2) a step of inducing aggregation of the nanoparticles through binding of the material coated on the surface of the nanoparticles and fibrinogen in a blood sample; (3) a step of measuring the spectroscopic property of the nanoparticles; and (4) a step of calculating fibrinogen concentration in the blood sample using the measured spectroscopic property of the nanoparticles.

In the present disclosure, the term “fibrinogen” is used to include natural fibrinogen, recombinant fibrinogen, or derivatives of fibrinogen that can be converted by thrombin to form fibrin (e.g., natural or recombinant fibrin monomers or derivatives that can or cannot self-assemble). Fibrinogen should be able to bind to at least two fibrinogen-binding peptides. The fibrinogen may be obtained from any source, and from any species (including bovine fibrinogen). But, specifically, it is human fibrinogen. Human fibrinogen can be autologous or can be obtained from the blood of a donor. Autologous fibrinogen or recombinant fibrinogen is preferred because the risk of infection when administered to a subject can be decreased.

In the present disclosure, the spectroscopic property of the nanoparticles may be absorbance in a particular wavelength range of irradiated light.

In the present disclosure, in the step of calculating fibrinogen concentration in the blood sample, the fibrinogen concentration may be calculated depending on a ratio of absorbance in a particular wavelength range where intensity is increased as the degree of aggregation of the nanoparticles is increased, and absorbance in a particular wavelength range where intensity is decreased as the degree of aggregation of the nanoparticles is increased.

In the present disclosure, the ratio of absorbance in a particular wavelength range where intensity is increased as the degree of aggregation of the nanoparticles is increased, and absorbance in a particular wavelength range where intensity is decreased as the degree of aggregation of the nanoparticles is increased was used to detect the fibrinogen concentration. This is for quantification and amplification of signals. Quantification using a single wavelength results in different absorbance values (arbitrary unit, a.u.) for different devices. This leads to different quantification results depending on measurement devices. In contrast, when two wavelengths are selected and the ratio of the different absorbance values is taken as in the present disclosure, a unitless constant result is obtained. Since the ratio is maintained constant even when different devices are used for quantification, significantly the same quantification result can be obtained regardless of the device used for absorbance measurement. In addition, since the absorbance in a particular wavelength range where intensity is increased is divided by the absorbance in a particular wavelength range where intensity is decreased, the signal is amplified as compared to when a single wavelength is selected.

In the present disclosure, the nanoparticle may be any one selected from a group consisting of a gold nanoparticle, a silver nanoparticle, a platinum nanoparticle, a silver nanocube, a silver nanoplate and a gold nanorod.

In the present disclosure, gold nanoparticles exhibit color change as they aggregate due to localized surface plasmon resonance (LSPR).

Accordingly, in the present disclosure, any nanomaterial exhibiting the LSPR phenomenon may be used as the nanoparticles. Examples include platinum nanoparticles, silver nanoparticles, gold nanoparticles, silver nanocubes, silver nanoplates, gold nanorods, etc.

Although gold nanoparticles used in the examples of the present disclosure as the nanoparticles, organic nanoparticles or non-organic nanoparticles such as inorganic nanoparticles, metal nanoparticles, etc. may also be used.

The gold nanoparticles used in the present disclosure are similar to those described in literatures [Schneider and Decher (Nano Letters, 2004, Vol. 4, No. 10, 1833-1839), Dorris et al. (Langmuir, 2008, 24(6), 2532-2538) and Schneider and Decher (Langmuir, 2008, 24, 1778-1789)]. Particles prepared from sodium polystyrene sulfonate are described in the literature of Chanana et al.

In an aspect of the present disclosure, gold nanoparticles coated with silica (silicon dioxide) may be used to increase stability.

In the present disclosure, the material may be a cell membrane.

In the present disclosure, the cell membrane may be a cell membrane of a red blood cell, a white blood cell (particularly, a monocyte or a macrophage) and a blood platelet. The cell membrane material may have fibrinogen receptors.

In the present disclosure, the cell membrane refers to a material capable of binding to fibrinogen. In the examples of the present disclosure, the cell membranes of red blood cells and monocytes were used.

In the present disclosure, the blood sample refers to whole blood, blood platelet-rich plasma and blood platelet-poor plasma. The blood sample may also refer to serum. For isolation according to the present disclosure to be possible under the condition described above, fibrinogen should be added to the sample. The blood sample according to the present disclosure may also be a blood substitute or an artificially prepared sample, composed of blood components, blood additives or other components mimicking the function of blood. Typical examples of blood components commonly used for blood transfusion include blood platelet concentrate, red blood cell (hemoglobin) concentrate, and serum or plasma substitute (also known as plasma volume expander). If the blood sample is deficient in a clotting factor (mainly fibrinogen), for example, such as a septic sample, a prepared blood sample or a blood substitute, the deficiency may be compensated for by adding a clotting factor including fibrinogen to the blood sample as an essential component for separating target particles or molecules according to the present disclosure.

Therefore, in the same context, the blood sample according to the present disclosure may also refer to an artificially prepared blood sample obtained by mixing a blood sample with a fibrinogen-deficient sample. The fibrinogen-deficient sample may include, for example, samples from any source such as biological, clinical, food and environmental samples. More particularly, the term blood sample according to the present disclosure includes an artificially prepared blood sample prepared by mixing clotting factors including at least fibrinogen with a fibrinogen-deficient sample.

In another aspect, the present disclosure relates to nanoparticles for the method for measuring fibrinogen concentration in a blood sample described above, wherein a material binding specifically to fibrinogen in a blood sample is coated on the surface of the nanoparticles.

In the present disclosure, the nanoparticles may aggregate as the material coated on the surface binds to fibrinogen.

In the present disclosure, the degree of aggregation of the nanoparticles may increase as the binding to fibrinogen is increased.

In the present disclosure, the nanoparticles may exhibit change in spectroscopic property depending on the degree of aggregation.

In the present disclosure, the nanoparticle may be any one selected from a group consisting of a gold nanoparticle, a silver nanoparticle, a platinum nanoparticle, a silver nanocube, a silver nanoplate and a gold nanorod.

In the present disclosure, the material may be a cell membrane.

In the present disclosure, the cell membrane may be a cell membrane of a red blood cell, a white blood cell (particularly, a monocyte or a macrophage) or a blood platelet. The cell membrane material may have fibrinogen receptors.

In the present disclosure, a “fibrinogen sensor” refers to cell membrane-coated nanoparticles.

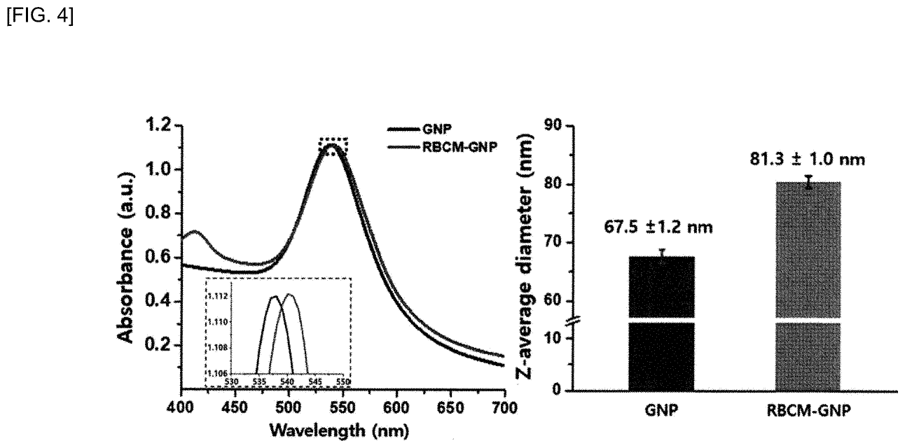

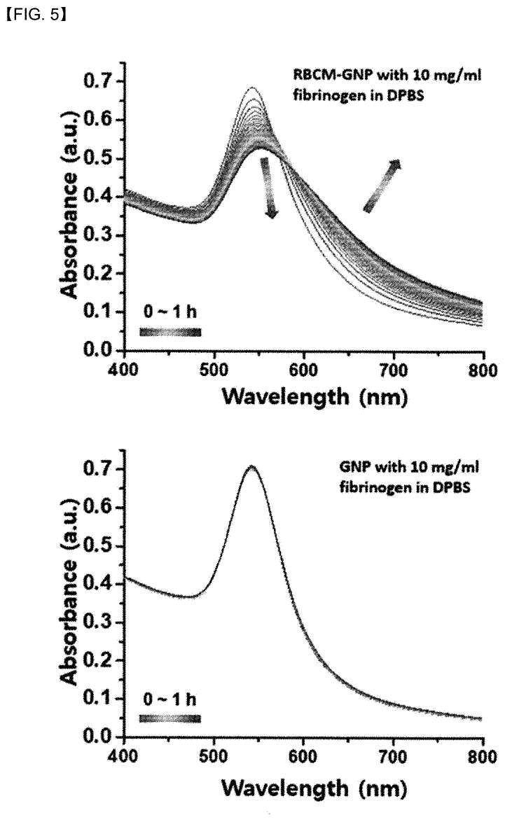

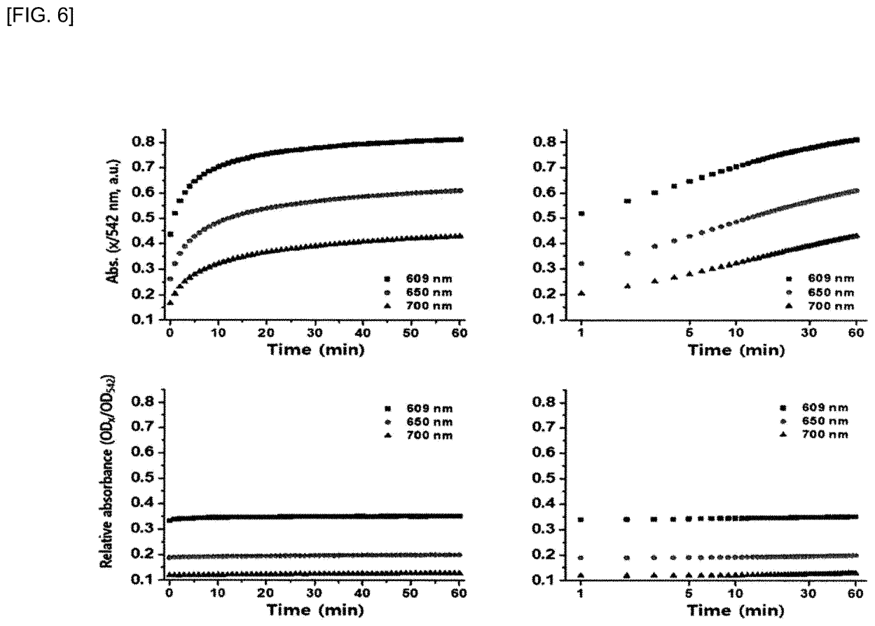

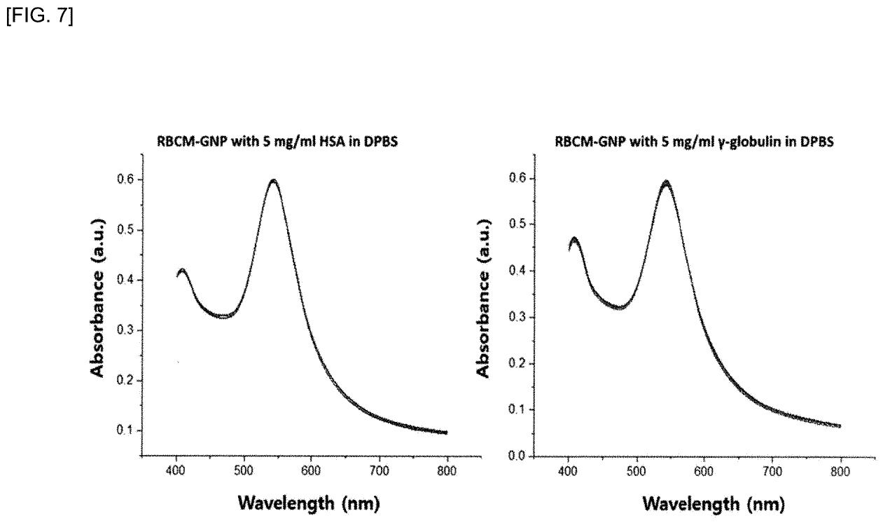

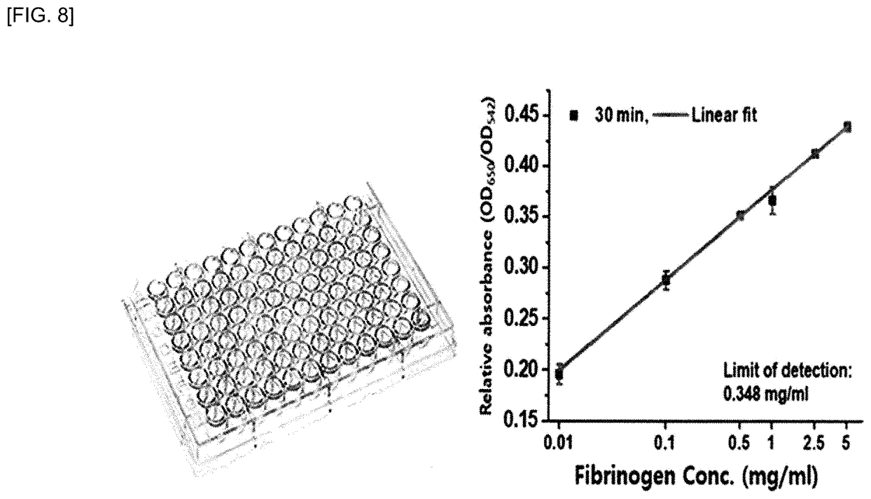

In an example of the present disclosure, red blood cell and monocyte membranes were purified and coated on gold nanoparticles. The change in the optical property and particle size of the red blood cell membrane-coated gold nanoparticles was identified (FIG. 4). Signal intensity was increased when the red blood cell membrane-coated gold nanoparticles were reacted with fibrinogen (FIG. 5 and FIG. 6). In contrast, when the same experiment was conducted on serum albumin and γ-globulin present in blood, it was confirmed that the two materials had no effect on the red blood cell membrane-coated gold nanoparticles (FIG. 7). In addition, through fibrinogen measurement using a multi-plate reader, it was confirmed that absorbance is increased as the fibrinogen concentration is increased due to aggregation of the fibrinogen sensors (FIG. 8).

In the present disclosure, the terms “purification” and “clarification” can be used interchangeably and refer to removal of impurities included in a re-dissolved solution obtained by re-dissolving precipitates, etc. in a buffer solution.

### MODE FOR INVENTION

Hereinafter, the present disclosure will be described in detail through examples. However, the following examples are for illustrative purposes only and it will be obvious to those of ordinary skill in the art that the scope of the present disclosure is not limited by the examples.

### Example 1: Preparation of Cell Membrane

1.1 Purification of Red Blood Cell Membrane

Blood (whole blood) was collected in a tube treated with EDTA. Then, the blood was centrifuged at 1000 g for 5 minutes while maintaining temperature at 4° C. After plasma, white blood cells, etc. and red blood cells were separated into upper and lower layers, respectively, the red blood cells were extracted from the blood by removing the upper layer. Then, the red blood cells were immersed in 1×PBS (pH 7.4, Gibco) and then extracted three times through centrifugation. Then, the red blood cells were immersed in 0.25×PBS for 20 minutes for hemolysis. In the PBS solution, red blood cell membrane, membrane proteins and hemoglobin exist together. In order to separate the cell membrane from the membrane proteins, the solution was centrifuged at 1000 g for 5 minutes. The, after removing the upper layer except for the light-pink red blood cell membrane and the membrane proteins that settled down in the lower layer, the remainder was washed three times.

1.2 Purification of Monocytes

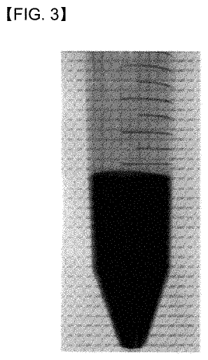

Cells and a cell culture medium were centrifuged at 1000 g for 5 minutes while maintaining temperature at 4° C. A population of cells was obtained by collecting the cells from the lower layer. The cells were immersed in 1×PBS (pH 7.4, Gibco) and then extracted three times through centrifugation. Then, the cells were immersed in 0.25×PBS for 20 minutes for hemolysis. In the PBS solution, cell membrane, membrane proteins and cell organelles exist together. In order to separate the cell membrane from the membrane proteins, the solution was centrifuged at 2000 g for 5 minutes. The, after removing the upper layer except for the red blood cell membrane and the membrane proteins that settled down in the lower layer, the remainder was washed three times (FIG. 3).

### Example 2: Coating of Cell Membrane on Nanoparticles

In order to coat the cell membrane on nanoparticles, the purified cell membrane (of red blood cells and monocytes) was diluted in purified water at a ratio of 1% (v/v) and then sonicated with an energy of 72 W for 5 minutes. The sonicated cell membrane (of red blood cells and monocytes) was in the form of spheres such as liposomes. After adding gold nanoparticles with a size of 50-100 nm thereto, the mixture was sonicated again for 5 minutes. The proportion of the nanoparticles and the cell membrane was 3 μL: 800 μL (0.025 mg/mL).

In the sonicated solution, the cell membrane (of red blood cells and monocytes) coated on the nanoparticles and the remaining cell membrane exist together. In order to remove the uncoated remaining cell membrane, centrifugation was performed at 3000 rpm for 50 minutes. After removing the upper layer except for the particles that settled down after the centrifugation, purified water of the same amount was added again.

As a result, red blood cell membrane-coated gold nanoparticles and monocyte-coated gold nanoparticles were obtained.

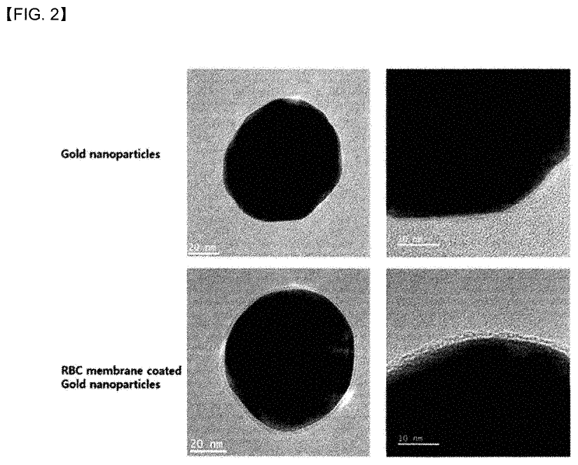

The gold nanoparticles and red blood cell membrane-coated gold nanoparticles were observed by TEM (FIG. 2).

### Example 3: Measurement of Fibrinogen Concentration

For measurement of fibrinogen concentration, fibrinogen of an appropriate concentration was dissolved in Dulbecco's phosphate buffer with calcium and magnesium and then measurement was made using a UV-Vis spectrometer and a multi-plate reader.

3.1 Measurement Using UV-Vis Spectrometer

After adding a mixture of 400 μL of particles and 400 μL of fibrinogen at a specific concentration in a transparent cuvette cell, measurement was made using a UV-Vis spectrometer in a range from 400 nm to 800 nm for 1 hour with 1-minute intervals. The signal at 650 nm divided by the signal at 542 nm was used as relative absorbance (A_{650 nm/542 nm}, A_{609 nm/542 nm} and A_{700 nm/542 nm}; A: absorbance).

As a result, it was confirmed that the coating with the red blood cell membrane resulted in increased reactivity to fibrinogen and increased signal intensity (FIG. 5 and FIG. 6).

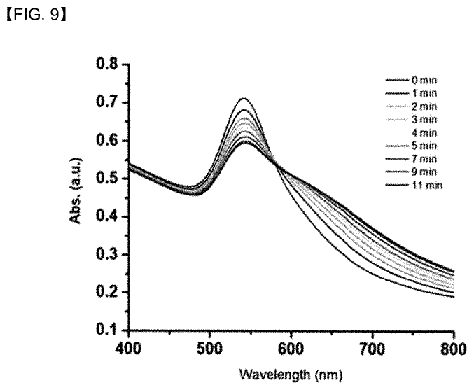

In addition, it was confirmed that the coating with the mononuclear leukocyte membrane also resulted in reaction with fibrinogen (FIG. 9).

Furthermore, when the same experiment was conducted on human serum albumin and γ-globulin, which are representative proteins present in blood, it was confirmed that the two materials had no effect on the sensor of the present disclosure (FIG. 7).

3.2 Measurement Using Multi-Plate Reader

After adding a mixture of 100 μL of particles and 100 μL of fibrinogen at a specific concentration in a transparent 96-well plate, measurement was made using a multi-plate reader at 542 nm and 650 nm at 25° C. for 30 minutes with 1-minute intervals. The signal at 650 nm divided by the signal at 542 nm was used as relative absorbance (A650 nm/542 nm). The use of the multi-plate reader enabled measurement of a large number of samples of smaller volumes at once.

As a result, it was confirmed that the relative absorbance is increased as the fibrinogen concentration is increased (FIG. 8).

While the specific embodiments of the present disclosure have been described in detail, it will be obvious to those of ordinary skill in the art that the specific embodiments are only preferred exemplary embodiments and the scope of the present disclosure is not limited by them. Accordingly, the substantial scope of the present disclosure is to be defined by the appended claims and their equivalents.

### INDUSTRIAL APPLICABILITY

The nanoparticles provided by the present disclosure make the use of enzyme and reference plasma for measurement of fibrinogen concentration unnecessary. Accordingly, fibrinogen concentration can be measured more conveniently and accurately with superior reproducibility. Therefore, it is expected that the present disclosure will be useful in the diagnosis market for evaluation of the risk of heart disease, evaluation of hereditary deficiency or anomaly of fibrinogen, etc.

1. Nanoparticles for measuring fibrinogen concentration, wherein the nanoparticle coated on the surface with a material, and wherein the material is specifically binds to fibrinogen.

2. The nanoparticles for measuring fibrinogen concentration according to claim 1, wherein the nanoparticles aggregate as the material coated on the surface binds to fibrinogen.

3. The nanoparticles for measuring fibrinogen concentration according to claim 1, wherein the degree of aggregation of the nanoparticles increases as the binding between the material coated on the surface and fibrinogen is increased.

4. The nanoparticles for measuring fibrinogen concentration according to claim 1, wherein the spectroscopic property of the nanoparticles changes depending on the degree of aggregation.

5. The nanoparticles for measuring fibrinogen concentration according to claim 1, wherein the nanoparticle is any one selected from a group consisting of a gold nanoparticle, a silver nanoparticle, a platinum nanoparticle, a silver nanocube, a silver nanoplate and a gold nanorod.

6. The nanoparticles for measuring fibrinogen concentration according to claim 1, wherein the material coated on the surface of the nanoparticles is a cell membrane.

7. The nanoparticles for measuring fibrinogen concentration according to claim 6, wherein the cell membrane is a cell membrane of one or more of a red blood cell, a white blood cell and a blood platelet.

8. A method for measuring fibrinogen concentration in a blood sample, comprising:
(1) a step of contacting nanoparticles having a material binding specifically to fibrinogen coated on the surface thereof with a blood sample;
(2) a step of inducing aggregation of the nanoparticles through binding of the material coated on the surface of the nanoparticles and fibrinogen in a blood sample;
(3) a step of measuring the spectroscopic property of the nanoparticles; and
(4) a step of calculating fibrinogen concentration in the blood sample using the measured spectroscopic property of the nanoparticles.

9. The method for measuring fibrinogen concentration in a blood sample according to claim 8, wherein the spectroscopic property measured in the step (3) is absorbance in a particular wavelength range absorbed by the nanoparticles.

10. The method for measuring fibrinogen concentration in a blood sample according to claim 8, wherein, in the step (4), the fibrinogen concentration is calculated using a ratio of absorbance in a particular wavelength range where intensity is increased as the degree of aggregation of the nanoparticles is increased, and absorbance in a particular wavelength range where intensity is decreased as the degree of aggregation of the nanoparticles is increased.

11. The method for measuring fibrinogen concentration in a blood sample according to claim 10, wherein the wavelength range where intensity is increased is 560-800 nm, and the wavelength range where intensity is decreased is 400-560 nm.

12. The method for measuring fibrinogen concentration in a blood sample according to claim 8, wherein the nanoparticle is any one selected from a group consisting of a gold nanoparticle, a silver nanoparticle, a platinum nanoparticle, a silver nanocube, a silver nanoplate and a gold nanorod.

13. The method for measuring fibrinogen concentration in a blood sample according to claim 8, wherein, in the step (1), the material coated on the surface of the nanoparticles is a cell membrane.

14. The method for measuring fibrinogen concentration in a blood sample according to claim 13, wherein the cell membrane is a cell membrane of one or more of a red blood cell, a white blood cell and a blood platelet.
